(19) United States
(12) Patent Application Publication
(10) Pub. No.: US 2011/0105722 A1
(43) Pub. Date: May 5, 2011

(54) Peptide synthesis method using n-carboxyanhydride (UNCA)

(75) Inventors: Roland Callens, Grimbergen (BE); Laurent Jeannin, Brussels (BE)

(73) Assignee: SOLVAY (SOCIETE ANONYME), Brussels (BE)

(21) Appl. No.: 12/921,413

(22) PCT Filed: Mar. 6, 2009
(86) PCT No.: PCT/EP09/52677

§ 371 Date: Sep. 8, 2010

(30) Foreign Application Priority Data
Mar. 10, 2008 (FR) 0851513

(51) Int. Cl. C07K 1/107; C07K 1/14

(52) U.S. Cl. 530/338

(57) ABSTRACT

Method for preparing a peptide or a peptide derivative which comprises it least one step in which a free amino acid or a free peptide is reacted with a urethane-protected amino acid N-carboxyanhydride (UNCA) solution.

The present invention relates to a method for synthesizing peptides or peptide derivatives.

Synthetic peptides have a large-scale application, especially as an active ingredient in medicinal products.

Peptide synthesis generally requires the use of amino acids which are both protected and activated prior to their use. This strategy is neither the simplest nor most economical.

Optionally protected amino acid N-carboxyanhydrides (hereinafter referred to as NCAs or Leuchs' anhydrides) are an advantageous alternative to conventional peptide couplings. NCAs, generally obtained by phosgenation of amino acids, are very reactive compounds that do not form, by rearrangement in particular, secondary products and the only reaction by-product of which is carbon dioxide. Amino acid N-carboxyanhydrides substituted by urethane groups (UNCAs) have been described in the literature, and in particular in the field of peptide synthesis.

More than a hundred UNCA derivatives have been described to date (see, for example, William D. Fuller et al., J. Am. Chem. Soc., 1990, 112, 7414-7416 and William D. Fuller et al., Urethane-protected alpha-amino acid N-carboxyanhydrides and peptide synthesis, Biopolymers, 1996, 40,183-205). It should be noted that only the amino acids having a primary amine functional group can be converted to their corresponding UNCA derivative.

William D. Fuller et al. reviewed (in Urethane-protected alpha-amino acid N-carboxyanhydrides and peptide synthesis, Biopolymers, 1996, 40,183-205) various applications of UNCAs in peptide synthesis, both in the solid phase and in the liquid phase, and commented on the advantages and disadvantages linked to their use.

The authors Zhu and Fuller described a rapid synthesis of tripeptides from dipeptide fragments having an ester-protected or amide-protected carboxyl functional group (Tetrahedron Letters, Vol. 36, No. 6, 807-810, 1995).

The object of the invention is, in particular, to provide an efficient, rapid and economical method for the synthesis of peptides or peptide derivatives having a high purity, especially high optical purity, and that can be easily used in industry. In particular, the method according to the invention makes it possible to carry out the large-scale synthesis of short peptides such as dipeptides, tripeptides or tetrapeptides.

The Applicant has found that the method according to the invention surprisingly makes it possible to obtain optically pure peptides with a high yield using free amino acids or free peptides in place of the amino acids or peptides protected on their carboxyl functional groups used previously. Moreover, the peptides or peptide derivatives obtained with the aid of the method do not generally require purification or, at the very least, can be easily purified.

The invention thus relates to a method for preparing a peptide or a peptide derivative which comprises at least one step in which a free amino acid or a free peptide is reacted with a urethane-protected amino acid N-carboxyanhydride (UNCA) solution.

The expression “amino acid” is understood to mean, for the purposes of the present invention, any compound comprising at least one NR₁R₂ group, which is preferably an NH₂ amine group, and at least one carboxyl group. The amino acids of the present invention may be of natural or synthetic origin. Natural amino acids, apart from glycine, contain a chiral carbon atom. The amino acids used in the present invention are preferably enantiopure amino acids. The expression “enantiopure amino acid” is understood to mean a chiral amino acid mainly composed of one enantiomer. The enantiomeric excess (ee) is defined as: ee (%)=100(x₁−x₂)/(x₁+x₂) with x₁>x₂; x₁ and x₂ represent the content of enantiomer 1 or 2 respectively in the mixture. It is possible to use natural or non-natural amino acids. Amino acids may have the D or L configuration. The residues of certain amino acids that can be used are abbreviated according to the following 3-letter codes: Alanine (Ala), Arginine (Arg), Aspartic acid (Asp), Asparagine (Asn), Cysteine (Cys), Glutamic acid (Glu), Glutamine (Gln), Glycine (Gly), Histidine (His), Isoleucine (Ile), Leucine (Leu), Lysine (Lys), Methionine (Met), Phenylalanine (Phe), Serine (Ser), Threonine (Thr), Tryptophane (Trp), Tyrosine (Tyr) and Valine (Val). Amino acids having nucleophilic side chains are advantageously protected in the side chain prior to their use in the method according to the present invention.

The expression “protecting group” is understood to mean any type of group that prevents the atom or the group to which it is attached, for example an oxygen or nitrogen atom, from participating in undesirable reactions during the synthesis. The protecting groups include side chain protecting groups and groups that protect the C- or N-terminal parts, commonly referred to as amine protecting groups and acid protecting groups.

As non-exhaustive examples of amine protecting groups, mention may be made, in particular, of benzoyl (Bz), acetyl (Ac), trifluoroacetyl (Tfa), benzyloxycarbonyl (Z), p-chlorobenzyloxycarbonyl (2C1Z), p-bromobenzyloxycarbonyl (2BrZ), p-nitrobenzyloxycarbonyl, p-methoxybenzyloxycarbonyl, benzhydryloxycarbonyl, 9-fluorenylmethyloxycarbonyl (Fmoc), tert-butyloxycarbonyl (Boc), benzenesulphonyl, p-toluenesulphonyl or 2-nitrobenzenesulphonyl groups.

As non-exhaustive examples of acid protecting groups, mention may be made of groups of alkyl, aryl, aralkyl or silyl type, such as methoxymethyl, methylthiomethyl, 2,2,2-trichloroethyl, 2-haloethyl, 2-(trimethylsilyl)ethyl, t-butyl, aryl, alkyl, aralkyl, allyl, benzyl, triphenylmethyl (trityl), benzhydryl, p-nitrobenzyl, p-methoxybenzyl and trialkylsilyl groups such as trimethylsilylethers, triethylsilyl, t-butyldimethylsilyl, or isopropyldimethylsilyl.

For the purposes of the present invention, the term “peptide” refers to a polymer in which the monomers are amino acids joined together by amide-type covalent bonds.

Peptide derivatives denote compounds analogous to the original peptides in which one or more atoms have been replaced or added. Typical examples of a peptide derivative may be selected from a peptide whose side groups are activated or protected, a peptide whose end groups are activated or protected, a cyclic form of a peptide or a peptide comprising a cyclic amino acid. The peptides comprise at least 2 amino acids. Preferably, the number of amino acids in the peptide chain is greater than or equal to 3. The peptide chain often comprises at most 100 amino acids. Preferably, the number of amino acids in the peptide chain is less than or equal to 20. Particularly preferably, the number of amino acids in the peptide chain is less than or equal to 15. The method according to the invention is particularly suitable for the synthesis, especially the large-scale synthesis, of dipeptides, tripeptides and tetrapeptides. It is also advantageous for producing, for example, pentapeptides, hexapeptides or heptapeptides.

Moreover, all the peptide sequences are represented by formulae ranging from the left to the right, the orientation of which is in the conventional direction, that is to say ranging from the amine terminal part to the carboxyl terminal part.

It has been found that the method according to the invention is particularly suitable for the synthesis of peptides and peptide derivatives exhibiting a high degree of diastereomeric purity.

The peptides and peptide derivatives obtained in the method according to the invention generally exhibit a diastereomeric purity, defined as desired diastereomer weight content, of greater than or equal to 98%. Often, the diastereomeric purity is greater than or equal to 99%. Preferably the diastereomeric purity is greater than or equal to 99.5%. Particularly preferably, the diastereomeric purity is greater than or equal to 99.9%.

The method according to the invention therefore enables the coupling of one amino acid in UNCA form with another free amino acid or a free peptide.

For the purposes of the invention, the term “coupling” refers, in particular, to the reaction between the carboxyl group of an amino acid or of the C-terminal part of a peptide and the amino group of another amino acid or the N-terminal end of a second peptide.

For the purposes of the invention, the term “C-terminal” denotes the terminal part or the end of the amino acid chain of a peptide terminated by a carboxyl (—COOH) group. Moreover, the term “N-terminal” refers to the terminal part or the end of the amino acid chain of a peptide terminated by an amino (—NH₂) group. For the purposes of the present invention, the free amino acid or the free peptide denotes an amino acid or a peptide having at least one carboxyl group, where appropriate C-terminal group, which is in the form of —COOH. More particularly, in the free amino acid or the free peptide the amino group, where appropriate N-terminal group, is in the form of —NH₂. More particularly still, “free amino acid” or “free peptide” denotes an unprotected amino acid or an unprotected peptide. It is understood that the internal salts of the free amino acids or peptides are, where appropriate, also included in this definition.

In the context of the present invention, the abbreviation “NCA” denotes an amino acid N-carboxyanhydride and “UNCA” denotes a urethane-protected amino acid N-carboxyanhydride.

The urethane-protected amino acid N-carboxyanhydride (UNCA) solution that reacts with the free amino acid or the free peptide in the method according to the present invention is generally obtained by dissolving the UNCA in a suitable solvent.

Preferably, the UNCA used in the method according to the invention is a UNCA that comprises a Boc, Fmoc or Z group. Most particularly preferably, the UNCA comprises a Boc group.

The protected peptide obtained when a protected UNCA is used may be deprotected and, if desired, used as a starting product for a following peptide synthesis step, in particular carried out according to the method according to the invention.

In the method according to the invention, advantageously a free amino acid or a free peptide is reacted with a UNCA solution in a solvent in which the free amino acid or the free peptide is at least partially soluble. Thus, the free amino acid in solid form or the free peptide in solid form may be brought into contact with the UNCA solution.

In the method according to the invention, the solvent is preferably chosen so that the free amino acid or the free peptide has a sufficient solubility in the solvent to initiate the reaction. Solvents are preferred that make it possible to attain a conversion to coupling product of at least 50% of the UNCA present in the solution in a reaction time less than or equal to 24 hours. More particularly, the solvent makes it possible to attain this conversion in a reaction time less than or equal to 12 hours, or even 6 hours.

More preferably, the solvent is a polar aprotic solvent that may especially be chosen from dimethylsulphoxide (DMSO), N,N-dimethylformamide (DMF), N,N-dimethylacetamide (DMA), N-methyl-2-pyrrolidone (NMP), formamide and sulpholane, tetrahydrofuran (THF) and acetonitrile. Excellent results have been obtained with dimethylsulphoxide.

As solvents, it is also possible to use ionic liquids, for example liquid salts of alkylated imidazoles.

In the method according to the invention, the reaction is generally carried out in a liquid medium. This medium may be homogeneous. Often, the reaction medium is, especially initially, heterogeneous, for example it may be a suspension of a free amino acid or of a free peptide in the UNCA solution. The reaction medium may also be composed of a solid substance composed of free amino acid or free peptide, which is immersed in the UNCA solution.

It is preferred that the liquid medium be substantially anhydrous. Generally, the water content in the liquid medium is kept at at most 1000 mg of water/kg of liquid medium. Often, this content is at most 500 mg of water/kg of liquid medium. Preferably, this content is at most 250 mg of water/kg of liquid medium. Often the water content in the liquid medium is greater than or equal to 10, or even 50, mg of water/kg of liquid medium.

When the method reacts a free amino acid and the UNCA solution, an enantiopure free amino acid is often used, that is to say a chiral amino acid mainly composed of one enantiomer, the enantiomeric excess of which is greater than or equal to 99%. An enantiopure amino acid having an enantiomeric excess greater than or equal to 99.5% is preferred. Particularly preferably, an enantiopure amino acid having an enantiomeric excess greater than or equal to 99.9% is used.

When the method reacts a free peptide and the UNCA solution, a diastereomerically pure free peptide is generally used, characterized by a diastereomeric purity greater than or equal to 98%. Often, the diastereomeric purity is greater than or equal to 99%. Preferably, the diastereomeric purity is greater than or equal to 99.5%. Particularly preferably, the diastereomeric purity is greater than or equal to 99.9%.

In the method according to the invention, the free amino acid or the free peptide is advantageously reacted with the UNCA solution, in proportions such that the free amino acid or the free peptide is in a slight stoichiometric excess with respect to the UNCA. Generally, from 1 to 1.5 equivalents of the free amino acid or the free peptide are used. Preferably, the amount of free amino acid or of free peptide used is greater than or equal to around 1.1 equivalents.

In the method according to the invention, the free amino acid or the free peptide is advantageously reacted with the UNCA solution at a temperature of 15° C. to 90° C. Often, the reaction is carried out at a temperature greater than or equal to 20° C. Preferably, the temperature is greater than or equal to 30° C. Often, the reaction is carried out at a temperature less than or equal to 80° C. Preferably, the temperature is less than or equal to 60° C.

The peptides and peptide derivatives obtained by the method according to the invention generally exhibit a diastereomeric purity, defined as desired diastereomer weight content, of greater than or equal to 98%. Often, the diastereomeric purity is greater than or equal to 99%. Preferably, the diastereomeric purity is greater than or equal to 99.5%. Particularly preferably, the diastereomeric purity is greater than or equal to 99.9%.

The pressure is generally chosen so as to keep the reaction medium, in particular the UNCA solution, in the liquid state.

According to one embodiment, atmospheric pressure (approximately 101.3 kPa) and superatmospheric pressures are particularly suitable.

According to another embodiment, pressures below atmospheric pressure are used. Often, in this embodiment, the pressure is equal to or higher than 400 mbar (40 kPa). Often, the pressure is equal to or lower than 500 mbar (50 kPa).

Atmospheric pressure (approximately 101.3 kPa) and especially pressures below atmospheric pressure are particularly suitable for eliminating the carbon dioxide formed by the reaction.

In the method according to the invention, the free amino acid or the free peptide is advantageously reacted with the UNCA solution over a reaction time which may range from 0.5 to 10 hours. Generally, this time is from 1 to 3 hours.

At the end of the reaction, unreacted free amino acid or unreacted free peptide may generally be recovered by a solid/liquid separation operation, for example centrifugation or, preferably, filtration. If necessary, in this embodiment, it may be advantageous to dilute the reaction medium with a second organic solvent, less polar than the solvent or mixture of solvents present in the reaction medium. By way of example of a suitable second solvent, mention may be made of alkyl esters, for example ethyl acetate or, preferably, isopropyl acetate.

If necessary after the recovery of the free amino acid or free peptide, the reaction medium is generally treated with water and the peptide or peptide derivative obtained may be recovered by extraction.

The peptide produced may be isolated, for example, by precipitation in a suitable precipitation solvent, typically an alkane, in particular chosen from cyclohexane, petroleum ether and n-heptane. It is also possible to isolate the peptide produced by formation of an ammonium salt, for example a salt of DCHA (dicyclohexylamine) or of CHA (cyclohexylamine).

The method according to the present invention makes it possible to obtain peptides and peptide derivatives with a yield typically greater than 80%.

The examples below are intended to illustrate the invention without, however, limiting it.

### EXAMPLE 1

### Synthesis of Boc-Ile-Leu-OH

45 ml of DMSO and also 6.30 g (1.2 eq.) of H-Leu-OH were introduced into a 250 ml round-bottomed flask in order to obtain a suspension. The reaction medium was brought to 60° C. before 10.29 g (1.0 eq.) of Boc-Ile UNCA were added thereto. After reacting for 2 hours, a sample from the reaction medium was analysed by HPLC. The reaction medium was cooled to ambient temperature before being diluted by 360 ml of isopropyl acetate.

0.1 eq. of dimethylaminopropylamine (DMAPA) was added and the reaction medium was stirred at ambient temperature for around 10 min.

The organic phase was then washed successively with:
300 ml of 5% aq. NaCl containing 1.0 eq. of KHSO₄ (5.45 g);
300 ml of 5 % aq. NaCl; and
300 ml of demineralized water.

The organic phase was concentrated by evaporation and an azeotropic drying operation was carried out with isopropyl acetate (300 ml in total). In the course of cooling, the dipeptide began to crystallize. It was diluted with 150 ml of cyclohexane and the evaporation was continued. The suspension was cooled to 0±5° C. After filtration, washing of the solid obtained with 70 ml of cyclohexane and drying, 12.8 g of the desired peptide were obtained.

Yield (based on NMR)=93%.

### EXAMPLES 2-6

A series of compounds were synthesized according to methods analogous to Example 1. The table below summarizes the results obtained for a series of tests carried out on the Boc-Ile UNCA.

Boc-Ile UNCA series

| Ex. | | | Purity | |
|---|---|---|---|---|
| No. | Structure | Conversion | NMR | Yield |
| 2 | Boc-Ile-Leu-OH | 100% | Peptide 100% | 93% |
| 3 | Boc-Ile-Pro-OH | 100% | Peptide 95% | 83% |
| | | | Isopropyl | |
| | | | acetate 1.2% | |
| 4 | Boc-Ile-Trp-OH•DCHA | 100% | Peptide 69% | 87% |
| | | | DCHA 30% | |
| 5 | Boc-Ile-Glu(OtBu)— | 100% | Peptide 68% | 71% |
| | OH•DCHA | | DCHA 29% | |
| 6 | Boc-Ile-Nle-Glu(OtBu)— | 100% | Peptide 73% | 71% |
| | OH | | DCHA 25.2% | |
| | | | DCU 0.6% | |
| | | | IPE 0.1% | |

1. A method for preparing a peptide or a peptide derivative, comprising at least one step in which a free amino acid or a free peptide is reacted with a urethane-protected amino acid N-carboxyanhydride (UNCA) solution.

2. The method according to claim 1, wherein the free amino acid or the free peptide is reacted with the UNCA solution in a solvent in which the free amino acid or the free peptide is at least partially soluble.

3. The method according to claim 2, wherein the solvent is a solvent in which the free amino acid or the free peptide has a solubility in the solvent that makes it possible to attain a conversion to coupling product of at least 50% of the UNCA present in the solution in a reaction time less than or equal to 24 hours.

4. The method according to claim 3, wherein the solvent is a polar aprotic solvent.

5. The method according to claim 4, wherein the polar aprotic solvent is chosen selected from the group consisting from dimethylsulphoxide (DMSO), N,N-dimethylformamide (DMF), N,N-dimethylacetamide (DMA), N-methyl-2-pyrrolidone (NMP), formamide and sulpholane, tetrahydrofuran (THF), and acetonitrile.

6. The method according to claim 5, wherein the solvent is DMSO.

7. The method according to claim 1, wherein the free amino acid or the free peptide is reacted with the UNCA solution at a temperature of from 15 to 90° C.

8. The method according to claim 1, wherein the UNCA comprises a Boc, Fmoc, or Z group.

9. The method according to claim 1, wherein carbon dioxide which is formed is drawn off.

10. The method according to claim 1, wherein the free amino acid or the free peptide is used in solid form.

11. The method according to claim 1, wherein the reaction medium is a suspension of free amino acid or of free peptide in the UNCA solution.

12. The method according to claim 1, wherein unreacted free amino acid or free peptide is recovered at the end of the reaction by a solid/liquid separation operation.

13. The method according to claim 12, wherein prior to the solid/liquid separation, the reaction medium is diluted with a second organic solvent, being less polar than the solvent or mixture of solvents present in the reaction medium.
